(19) United States
(12) Patent Application Publication
(10) Pub. No.: US 2004/0050679 A1
(43) Pub. Date: Mar. 18, 2004

(54) Method for producing (meth) acrylic acid

(76) Inventors: Ulrich Hammon, Mannheim (DE); Heinz Friedrich Sutoris, Worms (DE); Jurgen Schroder, Ludwigshafen (DE); Volker Schliephake, Schifferstadt (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/451,405

(22) PCT Filed: Dec. 21, 2001
(86) PCT No.: PCT/EP01/15206

(30) Foreign Application Priority Data
Dec. 22, 2000 (DE) 10064642.5

(51) Int. Cl. B01D 3/34; C23F 11/00; C23F 14/00; C01D 1/00; C07C 51/42; C07B 31/00

(52) U.S. Cl. 203/6; 203/8; 203/42; 203/49; 203/DIG. 1; 562/600

(57) ABSTRACT

(Meth)acrylic acid is worked up in the presence of at least one stabilizer by a process in which a stabilizer-containing mixture originating from the working-up and substantially freed from (meth)acrylic acid is passed into a distillation apparatus and a stabilizer-containing low boiler stream obtained from said apparatus is recycled to the working-up.

The present invention relates to a process for recycling stabilizers in the working-up of (meth)acrylic acid.

The use of stabilizers for reducing the polymerization of (meth)acrylic acid, i.e. methacrylic acid or acrylic acid, has long been known.

In Ullmann's Encyclopedia of Industrial Chemistry (6th ed, 1999 Electronic Release, chapter: Acrylic Acid and Derivatives—Acid Recovery and Purification), hydroquinone and hydroquinone monomethyl ether in the presence of oxygen are mentioned as suitable inhibitors for the polymerization of acrylic acid in distillation columns of the working-up stage.

The inhibitors used in the working-up process are incinerated with the residue.

Japanese Preliminary Published Application 53449/95 describes a process for inhibiting the polymerization of (meth)acrylic acid or (meth)acrylates, in which a combination of phenothiazine and hydroquinone with molecular oxygen is used in the distillation.

According to the last publication, the disadvantage of the prior art is that in general the polymerization inhibitors have to be added in large amounts in order for the polymerization to be reduced, particularly in the process steps in which (meth)acrylic acid is exposed to high temperatures.

Another disadvantage is that no possibility for recovering the stabilizer is mentioned.

As stated in Japanese Preliminary Published Application 53449/95, another unsatisfactory aspect of the prior art is that, when polymerization inhibitors having a relatively high vapor pressure are used, they readily mix with the product and discolor it.

PERP Report 96/97-8 “Acrylic Acid/Acrylates” of Chem Systems Inc., New York, 1997, pages 20 to 24, describes the recovery of a part of the inhibitor hydroquinone monomethyl ether used in the working up of acrylic acid, by extracting the high-boiling bottom discharge of the acrylic acid dimer cleavage optionally with water.

A disadvantage of this recovery process is the necessity of an additional process step for the extraction.

German laid-open application 2,901,783 describes a process for simultaneously recovering acrylic acid and hydroquinone, in which a tray liquid containing predominantly acrylic acid dimer, polymerization inhibitor and high-boiling sustances is subjected to vaporization with decomposition, in which the dimer or the acrylic acid eliminated therefrom acts as a flow medium and the inhibitor hydroquinone thus enters the distillate.

The remaining residue is additionally extracted with water to recover hydroquinone.

The vaporization with decomposition is carried out at from 120 to 220° C. and from 2 to 50 mbar.

The disadvantage of this process is that, in the examples, the hydroquinone content of the distillate is substantially lower than in the feed liquid. For effective recovery of the polymerization inhibitor, extraction with water is therefore necessary, constituting an additional process step.

Ullmann's Encyclopedia of Industrial Chemistry (6th ed, 1999 Electronic Release, chapter: Acrylic Acid and Derivatives—Esterification) describes processes for the preparation and working-up of higher or lower acrylates, in which the inhibitors used are recovered.

There, a part of the high-boiling, inhibitor-containing bottom discharge of the rectification column in which the product is separated from the high boilers is recycled in this form, together with other high boilers, into the working-up section or into the esterification reactor.

The other part of this bottom discharge, and if required a part-stream from the working-up section, is passed into a stripper in which high-boiling materials, such as inhibitors, impurities and polymers, are removed and incinerated in order to prevent their accumulation in the system.

The relatively large recycle streams, of which the inhibitor to be recycled accounts for only a small proportion and which reduce the capacity of the working-up are disadvantageous here. Furthermore, the inhibitor fed to the stripper is lost as a high boiler.

Furthermore, such bottom discharges often have a high viscosity, which makes them more difficult to pump.

In addition, it is known, for example from EP-A 765 856, that the polymerization of acrylic acid is accelerated by the presence of solid polymer so that recycling of a polymer-containing stream is to be avoided.

We have found that the consumption of stabilizer in a process for working up (meth)acrylic acid in the presence of at least one stabilizer can be reduced if a stabilizer-containing mixture originating from the working-up and substantially freed from (meth)acrylic acid is passed into a distillation apparatus and a stabilizer-containing low boiler stream obtained from said apparatus is recycled to the working-up.

In the context of this publication, stabilizers are those compounds which retard and/or inhibit the polymerization of (meth)acrylic acid. They may be, for example, inhibitors.

Here and below, polymerization means a substantially free radical polymerization of the (meth)acrylic acid, which is substantially irreversible.

Of course, the polymerization may also take place without free radicals, for example anionically or cationically.

Here, irreversible means that not more than 10, often less than 5, % by weight of the polymer used are cleaved into low-boiling components, for example oligomers or monomers, under the reaction conditions.

Polymers are those compounds which are formed by polymerization.

The novel process is in general carried out as follows:

A stream substantially freed from (meth)acrylic acid apart from a residual content and containing stabilizer (in active and/or inactive form), various high boilers of the (meth)acrylic acid preparation or working-up process, for example oligomers or polymers, and, if required, a solvent serving for absorption or extraction and referred to below as solvent is fed to a suitable distillation apparatus.

In this distillation apparatus, at least one stream containing stabilizer in its active form is separated from higher-boiling components and this lower-boiling stream is recycled to a suitable point of the working-up. Here, low boilers include the components which are removed in gaseous form in the distillation apparatus. In addition to stabilizer, the stream may also contain residual (meth)acrylic acid and/or solvent and further components, e.g. acrylic acid dimer.

The high boilers separated off can be subjected to this process several times more, disposed of, for example, by a thermal method, or, if required, fed to a thermal or catalytic cleavage, whose reaction discharge in turn can be subjected to the novel process or disposed of.

The residual (meth)acrylic acid content in the stream originating from the working-up and fed to the distillation apparatus is in general not more than 20, preferably not more than 10, particularly preferably not more than 5, % by weight.

The solvent may have been used in a preceding absorption and/or extraction and comprises the substances known to a person skilled in the art and usable for these purposes, for example water, methyl acrylate, ethyl acrylate, butyl acrylate, ethyl acetate, butyl acetate, biphenyl, diphenyl ether, dimethyl ortho-phthalate, diethyl ortho-phthalate, dibutyl ortho-phthalate or mixtures thereof.

A mixture of diphenyl ether and biphenyl, for example in the weight ratio of from 10:90 to 90:10, or a mixture to which from 0.1 to 25% by weight (based on the total amount of biphenyl and diphenyl ether) of at least one ortho-phthalate, e.g. dimethyl ortho-phthalate, diethyl ortho-phthalate or dibutyl ortho-phthalate, has also been added is preferably used.

The working-up of (meth)acrylic acid comprises the absorption, desorption, extraction and distillation steps which are known to a person skilled in the art for separating impurities from the gas mixture of the preparation of (meth)acrylic acid.

Suitable gas mixtures are those reaction discharges which are formed in the catalytic gas phase oxidation of C₃- or C₄-alkanes, -alkenes, -alkanols and/or -alkanals and/or precursors thereof to acrylic acid or methacrylic acid by known processes. Propene, propane, acrolein, tert-butanol, isobutene, isobutane, isobutyraldehyde, methacrolein, isobutyric acid or methyl tert-butyl ether is particularly advantageously used. However, other suitable starting compounds are those from which the actual C₃-/C₄- starting compound forms as an intermediate only during the gas-phase oxidation. Isobutyric acid and methyl tert-butyl ether may be mentioned by way of example for the preparation of methacrylic acid.

In the preparation of acrylic acid or methacrylic acid, as a rule the starting gases are diluted with inert gases, such as nitrogen, CO₂, saturated C₁-C₆-hydrocarbons and/or steam, mixed with oxygen and passed at elevated temperatures (usually from 200 to 450° C.) and, if required, superatmospheric pressure over transition metal mixed oxide catalysts, for example containing Mo and V or Mo, W, Bi and Fe, and oxidized to acrylic acid or methacrylic acid. These reactions are carried out, for example, in one or more stages. Particularly suitable processes for the preparation of methacrylic acid are those which start from methacrolein, in particular if the methacrolein is produced by gas-phase catalytic oxidation of tert-butanol, isobutane or isobutene or by reaction of formaldehyde with propionaldehyde.

The resulting reaction gas mixture contains, in addition to the desired acid, secondary components such as unconverted acrolein or methacrolein and/or propene or isobutene, steam, carbon monoxide, carbon dioxide, nitrogen, oxygen, acetic acid, propionic acid, formaldehyde, further aldehydes and maleic anhydride. In the case of acrylic acid, the reaction gas mixture usually contains, based in each case on the total reaction gas mixture, from 1 to 30% by weight of acrylic acid, from 0.01 to 1% by weight of propene and from 0.05 to 1% by weight of acrolein, from 0.05 to 10% by weight of oxygen, from 0.01 to 3% by weight of acetic acid, from 0.01 to 2% by weight of propionic acid, from 0.05 to 1% by weight of formaldehyde, from 0.05 to 2% by weight of other aldehydes, from 0.01 to 0.5% by weight of maleic acid and maleic anhydride and inert diluent gases as the remaining amount.

Such a gas mixture thus contains, in addition to the desired component (meth)acrylic acid which condenses predominantly as a medium boiling fraction which substantially comprises the components which have, at atmospheric pressure, a boiling point of, for example, from 120 to 180° C. in the case of acrylic acid, in particular in the range of +/−100° C. about that of the desired product, i.e. from about 131 to 151° C., further compounds in the high boiler and low boiler range and uncondensable components.

The working-up comprises, for example for acrylic acid, the absorption of the reactor discharge with one of the abovementioned solvents, e.g. biphenyl, diphenyl ether or a phthalate or a mixture thereof, or water, if necessary after cooling, for example by means of a quench or prequench. In the case of the absorption with water, an extraction of the acrylic acid with an organic solvent which may have a higher or lower boiling point than acrylic acid, e.g. ethyl acetate, butyl acetate, ethyl acrylate, biphenyl and/or diphenyl ether, follows.

If desired, the discharge of the absorption may be subjected to a desorption in which the discharge from the absorption is treated with gas in order to reduce the content of readily volatile components, e.g. acetaldehyde, propionaldehyde, acrolein or acetone.

In each of the abovementioned cases, distillative working-up usually follows, in which the acrylic acid is separated from troublesome impurities, secondary components or the solvent used. This may be effected in one or more stages.

The absorption in a solvent is not essential for the novel process, and a prior fractional condensation of a reaction mixture may also be used, as described, for example, in DE-A 197 40 253 or in the prior German application having the application number 100 53 086.9.

As stated at the outset, suitable measures to prevent polymer formation must be taken in the working-up, generally by using at least one stabilizer.

Usually, mostly high-boiling streams which contain the stabilizer or stabilizers used, generally in addition to polymers or oligomers, are obtained in this working-up.

Oligomers are those compounds which are formed by a Michael reaction of (meth)acrylic acid with other components present in the system, e.g. diacrylic acid (3-acryloyloxypropionic acid), tri- and tetracrylic acid, etc., di-, tri- and tetramethacrylic acid, etc., 3-hydroxypropionic acid and hydroquinone 2′-carboxyethyl ether.

Oligomers can generally be partially cleaved, i.e. to an extent of at least 20, preferably at least 30, % by weight, into lower-boiling components, for example by thermal treatment, in the presence or absence of a suitable catalyst, e.g. benzenesulfonic acid, para-toluenesulfonic acid, xylenesulfonic acid, dodecylbenzenesulfonic acid or sulfuric acid.

Such a conventional thermal and/or catalytic cleavage of oligomer-containing mixtures can of course also be combined with the novel process.

It is possible to use, as stabilizers for the novel process, one or more such compounds which are not sufficiently volatile to be separated off by distillation together with the (meth)acrylic acid, i.e. those stabilizers whose content in the vapor phase under the distillation conditions is not more than 20%, preferably not more than 10%, particularly preferably not more than 5%, of their content in the liquid phase fed in, but are sufficiently volatile to be separated in a suitable distillation step from high boilers present, i.e. those stabilizers whose content in the vapor phase under the distillation conditions is more than 5%, preferably at least 10%, particularly preferably at least 20%, of their content in the liquid phase fed in.

These may be, for example, N-oxyls, e.g. 4-hydroxy-2,2,6,6-tetramethylpiperidine-N-oxyl, 4-oxo-2,2,6,6-tetramethylpiperidine-N-oxyl, 4-acetoxy-2,2,6,6-tetramethylpiperidine-N-oxyl, 2,2,6,6-tetramethylpiperidine-N-oxyl, 4,4′,4″-tris(2,2,6,6-tetramethylpiperidine-N-oxyl) phosphite or 3-oxo-2,2,5,5-tetramethylpyrrolidin-N-oxyl, phenols and naphthols, e.g. p-aminophenol, p-nitrosophenol, 2-tert-butylphenol, 4-tert-butylphenol, 2,4-di-tert-butylphenol, 2-methyl-4-tert-butylphenol or 4-tert-butyl-2,6-dimethylphenol, quinones, e.g. hydroquinone or hydroquinone monomethyl ether, aromatic amines, e.g. N,N-diphenylamine, N-nitrosodiphenylamine, phenylenediamines, e.g. N,N′-dialkyl-para-phenylenediamine, where the alkyl radicals may be identical or different and, independently of one another, are each of 1 to 4 carbon atoms and may be straight-chain or branched, hydroxylamines, e.g. N,N-diethylhydroxylamine, urea derivatives, e.g. urea or thiourea, phosphorus-containing compounds, e.g. triphenylphosphine, triphenyl phosphite or triethyl phosphite, or sulfur-containing compounds, e.g. diphenyl sulfide.

Further suitable N-oxyls are 1-oxyl-2,2,6,6-tetramethyl-4-methoxypiperidine, 1-oxyl-2,2,6,6-tetramethyl-4-trimethylsilyloxypiperidine, 1-oxyl-2,2,6,6-tetramethylpiperidin-4-yl 2-ethylhexanoate, 1-oxyl-2,2,6,6-tetramethylpiperidin-4-yl stearate, 1-oxyl-2,2,6,6-tetramethylpiperidin-4-yl benzoate, 1-oxyl-2,2,6,6-tetramethylpiperidin-4-yl (4-tert-butyl)benzoate, bis(1-oxyl-2,2,6,6-tetramethylpiperidin-4-yl) succinate, bis(1-oxyl-2,2,6,6-tetramethylpiperidin-4-yl) adipate, bis(1-oxyl-2,2,6,6-tetramethylpiperidin-4-yl) 1,10-decanedicarboxylate, bis(1-oxyl-2,2,6,6-tetramethyl-piperidin-4-yl) n-butylmalonate, bis(1-oxyl-2,2,6,6-tetramethyl-piperidin-4-yl) phthalate, bis(1-oxyl-2,2,6,6-tetramethyl-piperidin-4-yl) isophthalate, bis(1-oxyl-2,2,6,6-tetramethyl-piperidin-4-yl) terephthalate, bis(1-oxyl-2,2,6,6-tetramethyl-piperidin-4-yl) hexahydroterephthalate, N,N′-bis(1-oxyl-2,2,6,6-tetramethylpiperidin-4-yl)adipamide, N-(1-oxyl-2,2,6,6-tetramethylpiperidin-4-yl)caprolactam, N-(1-oxyl-2,2,6,6-tetramethylpiperidin-4-yl)dodecylsuccinimide, 2,4,6-tris-[N-butyl-N-(1-oxyl-2,2,6,6-tetramethyl-piperidin-4-yl]triazine, N,N′-bis(1-oxyl-2,2,6,6-tetramethyl-piperidin-4-yl)-N,N′-bis-formyl-1,6-diaminohexane or 4,4′-ethylenebis(1-oxyl-2,2,6,6-tetramethylpiperazin-3-one).

Further suitable phenols and naphthols are o-, m- or p-cresol (methylphenol), 2-tert-butyl-4-methylphenol, 6-tert-butyl-2,4-dimethylphenol, 2,6-di-tert-butyl-4-methyl-phenol, 2,6-di-tert-butylphenol, 2,4-di-tert-butylphenol, 4-tert-butylphenol, nonylphenol[11066-49-2], bisphenol A, bisphenol F, bisphenol B, bisphenol S, bisphenol C, 3,3′,5,5′-tetrabromobisphenol A, octylphenol[140-66-9], 2,6-dimethylphenol, 2-methylhydroquinone, 2-(1′-methylcyclohex-1′-yl)-4,6-dimethyl-phenol, 2- or 4-(1′-phenyleth-1′-yl)phenol, 2,5-di-tert-butylhydroquinone, 2,5-di-tert-amylhydroquinone, 2,2′-methylenebis(6-tert-butyl-4-methylphenol), 2-tert-butyl-6-methylphenol, 2,4,6-tris-tert-butylphenol, 2,6-di-tert-butyl-p-cresol, 3,5-di-tert-butyl-4-hydroxyanisole, Koresin® from BASF AG, methyl 3,5-di-tert-butyl-4-hydroxybenzoate, 4-tert-butylpyrocatechol, 2-hydroxybenzyl alcohol, 2-methoxy-4-methylphenol, 2,3,6-trimethylphenol, 2,4,5-trimethylphenol, 2,4,6-trimethylphenol, 2-isopropylphenol, 4-isopropylphenol, 6-isopropyl-m-cresol, 4-tert-butyl-pyrocatechol, n-octadecyl β-(3,5-di-tert-butyl-4-hydroxy-phenyl)propionate, 1,1,3-tris-(2-methyl-4-hydroxy-5-tert-butyl-phenyl)butane, 1,3,5-trimethyl-2,4,6-tris(3,5-di-tert-butyl-4-hydroxybenzyl)benzene, 1,3,5-tris-(3,5-di-tert-butyl-4-hydroxybenzyl)isocyanurate, 1,3,5-tris(3,5-di-tert-butyl-4-hydroxyphenyl)propionyloxyethyl isocyanurate, 1,3,5-tris-(2,6-dimethyl-3-hydroxy-4-tert-butylbenzyl) isocyanurate or pentaerythrityl tetrakis [β-(3,5,-di-tert-butyl-4-hydroxyphenyl)propionate], 2,6-di-tert-butyl-4-dimethyl-aminomethylphenol, 6-sec-butyl-2,4-dinitrophenol, Irganoxe® 245, 259, 565, 1141, 1192, 1222, 1330, 1425 and MD1024 from Ciba Spezialitätenchemie, octadecyl 3-(3′,5′-di-tert-butyl-4′-hydroxy-phenyl)propionate, hexadecyl 3-(3′,5′-di-tert-butyl-4′-hydroxy-phenyl)propionate, octyl 3-(3′,5′-di-tert-butyl-4′-hydroxy-phenyl)propionate, 3-thia-1,5-pentanediol bis[(3′,5′-di-tert-butyl-4′-hydroxyphenyl)propionate], 4,8-dioxa-1,11-undecane-diol bis[(3′,5′-di-tert-butyl-4′-hydroxyphenyl)propionate], 1,7-heptanediamine-bis[3-(3′,5′-di-tert-butyl-4′-hydroxy-phenyl)propionamide], 3-(3′,5′-di-tert-butyl-4′-hydroxy-phenyl)propionic acid hydrazide, 3-(3′,5′-di-methyl-4′-hydroxy-phenyl)propionic acid hydrazide, bis(3-tert-butyl-5-ethyl-2-hydroxyphen-1-yl)methane, bis(3,5-di-tert-butyl-4-hydroxy-phen-1-yl)methane, bis[3-(1′-methylcyclohex-1′-yl)-5-methyl-2-hydroxyphen-1-yl]methane, bis(3-tert-butyl-2-hydroxy-5-methylphen-1-yl)methane, 1,1-bis(5-tert-butyl-4-hydroxy-2-methylphen-1-yl)ethane, bis(5-tert-butyl-4-hydroxy-2-methylphen-1-yl) sulfide, bis(3-tert-butyl-2-hydroxy-5-methyl-phen-1-yl) sulfide, 1,1-bis(3,4-dimethyl-2-hydroxyphen-1-yl)-2-methylpropane, 1,1-bis(5-tert-butyl-3-methyl-2-hydroxy-phen-1-yl)butane, 1,3,5-tris[1′-(3″,5″-di-tert-butyl-4″-hydroxyphen-1″-yl)meth-1′-yl]-2,4,6-trimethylbenzene, 1,1,4-tris(5′-tert-butyl-4′-hydroxy-2′-methylphen-1′-yl)butane, pyrocatechol (1,2-dihydroxybenzene), benzoquinone, 2-methyl-p-hydroquinone, 2,3-dimethylhydroquinone, trimethylhydroquinone, 3-methylpyrocatechol, 4-methylpyrocatechol, tert-butylhydroquinone, 2-methoxyphenol (guajacol, pyrocatechol monomethyl ether), 2-ethoxyphenol, 2-isopropoxyphenol, mono- or di-tert-butyl-4-methoxyphenol, 4-ethoxyphenol, 4-butoxyphenol, hydroquinone monobenzyl ether, p-phenoxyphenol, 4,4′-oxydiphenyl, 3,4-methylenedioxydiphenol (sesamol), 3,4-dimethylphenol, 3-hydroxy-4-methoxybenzyl alcohol, 2,5-dimethoxy-4-hydroxybenzyl alcohol (syringa alcohol), 4-hydroxy-3-methoxybenzaldehyde (vanillin), 4-hydroxy-3-ethoxybenzaldehyde (ethylvanillin), 3-hydroxy-4-methoxy-benzaldehyde (isovanillin), 1-(4-hydroxy-3-methoxyphenyl)ethanone (acetovanillone), eugenol, isoeugenol, dihydroeugenol or p-nitroso-o-cresol.

Other suitable quinones are p-benzoquinone, tetramethyl-p-benzoquinone, diethyl-1,4-cyclohexane-dione-2,5-dicarboxylate, phenyl-p-benzoquinone, 2,5-dimethyl-3-benzyl-p-benzoquinone, 2-isopropyl-5-methyl-p-benzoquinone (thymoquinone), 2,6-diisopropyl-p-benzoquinone, 2,5-dimethyl-3-hydroxy-p-benzoquinone, 2,5-dihydroxy-p-benzoquinone, embelin, tetrahydroxy-p-benzoquinone, 2,5-dimethoxy-1,4-benzoquinone, 2-amino-5-methyl-p-benzoquinone, 2,5-bisphenylamino-1,4-benzoquinone, 5,8-dihydroxy-1,4-naphtho-quinone, 2-anilino-1,4-naphthoquinone, anthraquinone, N,N-dimethylindoaniline, N,N-diphenyl-p-benzoquinonediimine, 1,4-benzoquinone dioxime, coerulignone, 3,3′-di-tert-butyl-5,5′-dimethyldiphenoquinone, p-rosolic acid and 2,6-di-tert-butyl-4-benzylidenebenzoquinone.

Examples of further suitable aromatic amines are nitrosodiethylaniline and Irganox 5057 from Ciba Spezialitätenchemie.

Further suitable phenylenediamines are N,N′-diisobutyl-p-phenylenediamine, N,N′-diisopropyl-p-phenylendiamine, p-phenylenediamine, N-phenyl-p-phenylenediamine, N,N′-diphenyl-p-phenylenediamine, N-isopropyl-N-phenyl-p-phenylenediamine, N,N′-di-sec-butyl-p-phenylenediamine (Kerobit® BPD from BASF AG), N-phenyl-N′-isopropyl-p-phenylenediamine (Vulkanox® 4010 from Bayer AG), N-(1,3-dimethylbutyl)-N′-phenyl-p-phenylenediamine, diphenylamine, N-phenyl-2-naphthylamine, iminodibenzyl, N,N′-diphenylbenzidine, N-phenyltetraaniline, acridone, 3-hydroxydiphenylamine and 4-hydroxydiphenylamine.

Tocopherols are furthermore suitable, e.g. α-, β-, γ-, δ- and ε-tocopherol, tocol, α-tocopherolhydroquinone, and 2,3-dihydro-2,2-dimethyl-7-hydroxybenzofuran (2,2-dimethyl-7-hydroxycoumaran).

They may also be decomposition products or derivatives of stabilizers, for example the Michael adduct of (meth)acrylic acid and hydroquinone.

Particularly suitable stabilizers are those whose vapor pressures at 141° C. (boiling point of acrylic acid) at atmospheric pressure are at least 15, preferably from 20 to 800, particularly preferably from 25 to 500, very particularly preferably from 25 to 250, in particular from 25 to 160, hPa and mixtures thereof.

These stabilizers can of course also be used as a mixture with virtually nonvolatile stabilizers, e.g. phenothiazine or inorganic salts, such as copper salts, e.g. copper dimethyldithiocarbamate, copper diethyldithiocarbamate, copper dibutyldithiocarbamate or copper salicylate, or manganese salts, e.g. manganese acetate.

Stabilizers as described in the contemporary application “Preparation of acrylic acid” of BASF AG with the same date of filing as the present application with the German Application number 100 64 641.1 are particularly suitable, i.e. stabilizer combinations containing phenothiazine and at least one phenolic compound and, if required, at least one metal salt, which are introduced at the top or the region of the top of the rectification or condensation column.

The fresh stabilizer added may be introduced in each case individually or as a mixture, in liquid or solid form or as a solution in a suitable solvent.

The method of addition of the fresh, i.e. unrecycled, stabilizer is not limited.

The addition can be effected at any desired point of the working-up process, for example with the quench liquid in the prequench or quench, in an absorption column, for example with the absorbent, with the feed or at any desired point of the absorption column, or in a distillation column, for example with the feed, with the reflux, in a cooling or quench circulation or directly into the column or at a plurality of points, as described, for example, in WO 00/20594.

If a plurality of different stabilizers are added they may be introduced independently of one another at different points of the working-up process. The addition of the stabilizer in a distillation or absorption is preferred, such stabilizer particularly preferably being metered into a distillation and very particularly preferably into a cooling or quench circulation of a distillation or being metered in directly.

The stabilizer added substantially reduces the free radical polymerization.

In general, stabilizers can preferably act on those radicals where the free radical center is present at an oxygen atom, for example hydroquinone or hydroquinone monomethyl ether, or on those where the free radical center is present at a carbon atom, for example in the case of N-oxyls or aromatic amines, or they can preferably decompose, for example, peroxides, e.g. phosphorus- or sulfur-containing stabilizers.

The process can be carried out in the presence or absence of molecular oxygen (O₂). If it is carried out in the presence of oxygen, it is possible to use an oxygen-containing gas in which the oxygen is, if required, diluted with an inert gas, such as carbon dioxide (CO₂), nitrogen (N₂) or a noble gas, e.g. helium or argon, or a mixture thereof. Nitrogen is preferably used for the dilution.

The oxygen content of the oxygen-containing gas may be from 1 to 100, preferably from 2 to 80, particularly preferably from 5 to 50, in particular from 10 to 30, % by volume.

In a preferred embodiment, air is used as the oxygen-containing gas.

If the process is carried out in the absence of oxygen, any of the abovementioned inert gases may be used individually or as a mixture. However, the process is preferably carried out in the presence of an oxygen-containing gas.

Those working-up streams in which the stabilizer is present at least partly in its polymerization-retarding and/or polymerization-inhibiting form, referred to here as the active form, are suitable for the novel treatment. Those working-up streams in which the proportion of the stabilizer in its active form is at least 10, particularly preferably at least 25, very particularly preferably at least 50, % by weight, based on the total amount of stabilizer in its active and in its inactive form, are particularly suitable.

These may be working-up streams which are removed directly or indirectly from the bottom discharge, side take-off, natural circulation, forced circulation or pumped circulation of a distillation, stripping or rectification apparatus, an absorber or desorber or a quench apparatus. It is of course also possible to use mixtures thereof, if required after storage.

The content of (active and inactive) stabilizer in the stream fed to the distillation apparatus is in general from 1 to 50 000, preferably from 5 to 30 000, particularly preferably from 20 to 20 000, very particularly preferably from 50 to 10 000, in particular from 100 to 5 000, ppm (w/w).

The residual content of (meth)acrylic acid in the stream fed to the distillation apparatus may be, for example, up to 50, preferably up to 40, particularly preferably up to 25, very particularly preferably up to 10, % by weight.

Suitable distillation apparatuses for the novel process are all distillation apparatuses known to a person skilled in the art, for example circulation evaporators, thin-film evaporators, falling-film evaporators, wiper-blade evaporators, if required in each case with attached rectification columns, and stripping columns.

In addition, it is possible to establish a stripping stream, for example with nitrogen or an oxygen-containing gas, preferably with air.

The distillation apparatus can be operated at from 80 to 350° C., preferably from 100 to 280° C., particularly preferably from 120 to 200° C., in particular from 130 to 200° C., and, independently thereof, at from 5 to 2 000, preferably from 5 to 1 200, particularly preferably from 20 to 500, very particularly preferably from 50 to 400, in particular from 50 to 300, hPa.

The stabilizer content of the low boiler stream recycled from the distillation apparatus into the working-up is in general at least 30%, preferably at least 40%, particularly preferably at least 50%, very particularly preferably at least 60%, in particular at least 85%, of the stabilizer content of the stream fed to the distillation apparatus.

Suitable points for recycling the stream obtained from the distillation apparatus are those steps of the working-up process in which the (meth)acrylic acid mixture to be worked up and present as a gaseous or liquid phase is heated, i.e. is exposed to temperatures above, for example, 80° C., in particular above 100° C., very particularly above 150° C. These are, for example, absorption, desorption, extraction, heating, evaporation distillation, rectification or condensation, for example in evaporators, distillation apparatuses, condensers, quench apparatuses, absorbers, desorbers and reflux dividers.

In a particularly preferred embodiment, a gaseous stream from a thermal and/or catalytic cleavage of a high boiler stream is recycled in the same manner as a stream obtained from the distillation apparatus described. A high boiler stream suitable for the cleavage may be, for example, the high boiler discharge of a condensation or rectification column or of the above-described distillation apparatus for the novel process.

The temperature during the cleavage is in general from 100 to 220° C., preferably from 120 to 200° C., particularly preferably from 140 to 180° C., in particular from 150 to 180° C.

The removal of the low boilers can be supported by passing through a gas stream which is substantially inert under the reaction conditions (stripping), e.g. nitrogen or steam, or an oxygen-containing gas, such as air.

Some of the remaining residue can, for example, be discharged, distilled or subjected to a cleavage again.

The gaseous stream which is obtained from the cleavage and may also contain stabilizer in addition to low-boiling cleavage products, e.g. acrylic acid, methacrylic acid or diacrylic acid, can be recycled to any desired point of the working-up process, for example to the quench or prequench or absorption, rectification or condensation column, if necessary after condensation and/or further cooling.

The novel process permits improved cost-efficiency through a reduced consumption of stabilizer in the working-up of (meth)acrylic acid, since stabilizer which is still active is not disposed of, as has been the case to date but is reused in the working-up, so that this process has not only an economic advantage but also an ecological one.

The examples which follow illustrate the novel process.

### COMPARATIVE EXAMPLES 1-3

Acrylic acid containing 150 ppm by weight of hydroquinone monomethyl ether is distilled in a Sambay evaporator in such a way that, at the top pressure stated in the table, the gas phase has a temperature as stated in the table. The gas phase is discharged, completely condensed and analyzed. A hydroquinone monomethyl ether (MEHQ) content as stated in the table is found.

| Comp. | Temperature | Top pressure | MEHQ content |
|---|---|---|---|
| example | [° C.] | [hPa] | [ppm] |
| 1 | 100 | 241 | 2 |
| 2 | 130 | 711 | 3 |
| 3 | 180 | 3 003 | 4 |

The experiments show that hydroquinone monomethyl ether does not substantially pass over with acrylic acid under conventional distillation conditions.

### EXAMPLES 1-3

A mixture consisting of 75% by weight of diphenyl ether and 25% by weight of biphenyl and containing 150 ppm by weight of hydroquinone monomethyl ether is distilled at the same temperature and in the same apparatus as in comparative examples 1 to 3. The following contents are obtained in the condensed gas phase:

| Ex- | Temperature | Top pressure | MEHQ content |
|---|---|---|---|
| ample | [° C.] | [hPa] | [ppm] |
| 1 | 100 | 5 | 115 |
| 2 | 130 | 21 | 130 |
| 3 | 180 | 131 | 165 |

The examples show that hydroquinone monomethyl ether can be distilled with a high-boiling solvent.

### EXAMPLES 4-6

A mixture comprising 99% by weight of a mixture of 75% by weight of diphenyl ether and 25% by weight of biphenyl, 1% by weight of acrylic acid and 150 ppm by weight of hydroquinone monomethyl ether is distilled at the same temperature and in the same apparatus as in examples 1 to 3. The following contents are obtained in the condensed gas phase:

| | | | | Acrylic acid |
|---|---|---|---|---|
| | Temperature | Top pressure | MEHQ content | content |
| Example | [° C.] | [hPa] | [ppm] | [% by weight] |
| 4 | 100 | 16 | 52 | 53.1 |
| 5 | 130 | 55 | 71 | 43.7 |
| 6 | 180 | 275 | 106 | 34.0 |

The examples show that hydroquinone monomethyl ether can be distilled together with acrylic acid from a high-boiling solvent.

### COMPARATIVE EXAMPLES 4-6

A mixture comprising 99% by weight of a mixture of 75% by weight of diphenyl ether and 25% by weight of biphenyl, 1% by weight of acrylic acid and 150 ppm by weight of phenothiazine (PTZ) is distilled at the same temperature and in the same apparatus as in examples 1 to 3. The following contents are obtained in the condensed gas phase:

| | | | | Acrylic acid |
|---|---|---|---|---|
| Comp. | Temperatur | Top pressure | PTZ content | content |
| example | [° C.] | [hPa] | [ppm] | [% by weight] |
| 4 | 100 | 16 | 0 | 53.1 |
| 5 | 130 | 55 | 0 | 43.7 |
| 6 | 180 | 275 | 0 | 34.0 |

Comparative examples 4-6 show that phenothiazine cannot be distilled together with acrylic acid from a high-boiling solvent.

### EXAMPLE 7

2 ml of acrylic acid and 20 ppm by weight of phenothiazine are filled/together with the amount of hydroquinone monomethyl ether stated in the table, into a glass ampoule in an air atmosphere, closed gas-tight and incubated at 120° C. in an oil bath at a depth of immersion of 4 cm. The time t to complete polymerization is determined.

The ratio of time t(PTZ+MEHQ) to complete polymerization of phenothiazine/hydroquinone monomethyl ether mixture to phenothiazine (20 ppm) alone t(PTZ) indicates the relative efficiency: t(PTZ+MEHQ)/t(PTZ)

| MEHQ (ppm) | t(PTZ + MEHQ)/t(PTZ) |
|---|---|
| 0 | 1 |
| 5 | 1.15 |
| 10 | 1.17 |
| 20 | 1.43 |
| 30 | 1.5 |
| 40 | 1.78 |
| 60 | 1.87 |
| 80 | 2.08 |
| 100 | 2.11 |
| 140 | 2.91 |
| 160 | 3.41 |
| 180 | 3.76 |
| 200 | 4.05 |

It is clear that the efficiency of phenothiazine in acrylic acid can be increased by adding hydroquinone monomethyl ether.

We claim:

1. A process for working up (meth)acrylic acid in the presence of at least one stabilizer, wherein a stabilizer-containing mixture originating from the working-up and substantially freed from (meth)acrylic acid is passed into a distillation apparatus and a stabilizer-containing low boiler stream obtained from said apparatus is recycled to the working-up.

2. A process as claimed in claim 1, wherein the distillation apparatus is operated at from 80 to 350° C.

3. A process as claimed in claim 1 or 2, wherein the distillation apparatus is operated at from 5 to 2 000 hPa.

4. A process as claimed in any of claims 1 to 3, wherein the stabilizer content in the low boiler stream recycled from the distillation apparatus to the working-up is at least 30% of the stabilizer content of the stream fed to the distillation apparatus.

5. A process as claimed in any of claims 1 to 4, wherein the stabilizer used is at least one compound from the group consisting of hydroquinone and hydroquinone monomethyl ether.

6. A process as claimed in any of claims 1 to 5, wherein the stabilizer-containing mixture originating from the working-up contains at least one compound from the group consisting of biphenyl, diphenyl ether and dimethyl ortho-phthalate.

7. A process as claimed in any of claims 1 to 6, wherein the stabilizer-containing mixture originating from the working-up contains not more than 20% by weight of (meth)acrylic acid.

8. A process as claimed in any of claims 1 to 7, wherein the working-up is carried out in the presence of an oxygen-containing gas.

9. A process as claimed in any of claims 1 to 8, wherein from 1 to 10 000 ppm of stabilizer are present in the mixture fed to the distillation apparatus.

10. A process as claimed in any of claims 1 to 9, wherein the stream obtained in the distillation apparatus is fed to an absorption step into which the (meth)acrylic acid-containing reaction mixture obtained from the oxidation is passed, if required after cooling.
